(19) United States
(12) Patent Application Publication
(10) Pub. No.: US 2019/0184056 A1
(43) Pub. Date: Jun. 20, 2019

(54) METHOD FOR USING AN ADHESIVE COMPOSITION IN BONE PRESERVATION AND AUGMENTATION

(71) Applicant: MANAR M. JAMAL, Pomona, CA (US)

(72) Inventor: MANAR M. JAMAL, Pomona, CA (US)

(21) Appl. No.: 15/844,450

(22) Filed: Dec. 15, 2017

(51) Int. Cl. A61L 24/00; A61L 24/02; A61L 27/38; A61L 27/36; A61L 27/12; A61L 27/10; A61L 27/02; A61L 27/54; A61L 27/56; A61L 27/58; A61L 24/10; A61L 27/24; A61C 13/15; A61F 2/28; A61C 8/02

(52) U.S. Cl. CPC A61L 24/0036; A61F 2002/3093; A61L 24/0042; A61L 24/0005; A61L 24/02; A61L 27/3834; A61L 27/3608; A61L 27/12; A61L 27/10; A61L 27/025; A61L 27/54; A61L 27/56; A61L 27/58; A61L 27/3847; A61L 27/3821; A61L 27/365; A61L 24/102; A61L 27/24; A61C 19/003; A61F 2/2857; A61C 8/0006; A61L 2300/414; A61L 2430/02; A61L 2300/64; A61L 2300/604; A61L 2430/12; A61L 2430/34; A61F 2250/0081; A61F 2002/2835; A61L 24/0015

(57) ABSTRACT

A method comprising the steps of applying an adhesive composition to a substrate bone repair region. The adhesive composition comprises a porous, biocompatible, biodegradable, resorbable, non-toxic, and polymerizable composition. The substrate bone repair region comprises a bone structure, implant or device surface, or tooth surface. The adhesive composition is cured to provide a polymerized adhesive composition resulting in repairing or augmenting the substrate bone repair region. The adhesive composition may be mixed with a bone graft material before application. The adhesive composition may be additionally applied to a barrier membrane that is placed over the substrate bone repair region where the adhesive composition or combination of adhesive composition and bone graft material is applied.

### COPYRIGHT NOTICE

A portion of the disclosure of this patent document contains material that is subject to copyright protection by the author thereof. The copyright owner has no objection to the facsimile reproduction by anyone of the patent document or patent disclosure for the purposes of referencing as patent prior art, as it appears in the Patent and Trademark Office, patent file or records, but otherwise reserves all copyright rights whatsoever.

### BACKGROUND OF THE RELEVANT PRIOR ART

One or more embodiments of the invention generally relate to a method for bone preservation. More particularly, certain embodiments of the invention relate to a method for using an adhesive composition in bone preservation, in bone regeneration to repair defects in bones, and in bone augmentation.

The following background information may present examples of specific aspects of the prior art (e.g., without limitation, approaches, facts, or common wisdom) that, while expected to be helpful to further educate the reader as to additional aspects of the prior art, is not to be construed as limiting the present invention, or any embodiments thereof, to anything stated or implied therein or inferred thereupon. The following is an example of a specific aspect in the prior art that, while expected to be helpful to further educate the reader as to additional aspects of the prior art, is not to be construed as limiting the present invention, or any embodiments thereof, to anything stated or implied therein or inferred thereupon. By way of educational background, another aspect of the prior art generally useful to be aware of is that a typical approach followed in bone preservation, bone regeneration and bone augmentation may include placing powdered, granules, putty, gel, or block bone graft materials over a bone and or around an implant or a device or a tooth covered with either resorbable barrier membrane or non-resorbable barrier membrane. However there may be an omnipresent danger of movement of the resorbable membrane during the healing period and movement and leakage of the bone graft materials and loss of the space volume required for bone regeneration, or the required fixation of preformed block bone graft material and non-resorbable membrane with surgical screws, or the need for a second surgery to remove non resorbable membrane, in addition to wound dehiscence, wound infection, increasing patient discomfort and the risk of losing some of the regenerated bone among others.

In view of the foregoing, it is clear that these traditional techniques are not perfect and leave room for more optimal approaches.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 illustrates perspective views of substrate bone repair regions, in accordance with an embodiment of the present invention; and

FIG. 2 illustrates a method of applying and curing an adhesive composition on a substrate bone repair region, in accordance with an embodiment of the present invention;

FIG. 3 illustrates a method of applying and curing an adhesive composition combined with a bone graft material on a substrate bone repair region, in accordance with an embodiment of the present invention; and

FIG. 4 illustrates a method applying and curing an adhesive composition on a barrier membrane which is placed on a substrate bone repair region where the adhesive composition is applied and cured, in accordance with an embodiment of the present invention.

Unless otherwise indicated illustrations in the figures are not necessarily drawn to scale.

### DETAILED DESCRIPTION OF SOME EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

All words of approximation as used in the present disclosure and claims should be construed to mean “approximate,” rather than “perfect,” and may accordingly be employed as a meaningful modifier to any other word, specified parameter, quantity, quality, or concept. Words of approximation, include, yet are not limited to terms such as “substantial”, “nearly”, “almost”, “about”, “generally”, “largely”, “essentially”, “closely approximate”, etc.

As will be established in some detail below, it is well settle law, as early as 1939, that words of approximation are not indefinite in the claims even when such limits are not defined or specified in the specification.

For example, see Ex parte Mallory, 52 USPQ 297, 297 (Pat. Off. Bd. App. 1941) where the court said “The examiner has held that most of the claims are inaccurate because apparently the laminar film will not be entirely eliminated. The claims specify that the film is “substantially” eliminated and for the intended purpose, it is believed that the slight portion of the film which may remain is negligible. We are of the view, therefore, that the claims may be regarded as sufficiently accurate.”

Note that claims need only “reasonably apprise those skilled in the art” as to their scope to satisfy the definiteness requirement. See Energy Absorption Sys., Inc. v. Roadway Safety Servs., Inc., Civ. App. 96-1264, slip op. at 10 (Fed. Cir. Jul. 3, 1997) (unpublished) Hybridtech v. Monoclonal Antibodies, Inc., 802 F.2d 1367, 1385, 231 USPQ 81, 94 (Fed. Cir. 1986), cert. denied, 480 U.S. 947 (1987). In addition, the use of modifiers in the claim, like “generally” and “substantial,” does not by itself render the claims indefinite. See Seattle Box Co. v. Industrial Crating & Packing, Inc., 731 F.2d 818, 828-29, 221 USPQ 568, 575-76 (Fed. Cir. 1984).

Moreover, the ordinary and customary meaning of terms like “substantially” includes “reasonably close to: nearly, almost, about”, connoting a term of approximation. See In re Frye, Appeal No. 2009-006013, 94 USPQ2d 1072, 1077, 2010 WL 889747 (B.P.A.I. 2010) Depending on its usage, the word “substantially” can denote either language of approximation or language of magnitude. Deering Precision Instruments, L.L.C. v. Vector Distribution Sys., Inc., 347 F.3d 1314, 1323 (Fed. Cir. 2003) (recognizing the “dual ordinary meaning of th[e] term [”substantially“] as connoting a term of approximation or a term of magnitude”). Here, when referring to the “substantially halfway” limitation, the Specification uses the word “approximately” as a substitute for the word “substantially” (Fact 4). (Fact 4). The ordinary meaning of “substantially halfway” is thus reasonably close to or nearly at the midpoint between the forwardmost point of the upper or outsole and the rearwardmost point of the upper or outsole.

Similarly, the term ‘substantially’ is well recognized in case law to have the dual ordinary meaning of connoting a term of approximation or a term of magnitude. See Dana Corp. v. American Axle & Manufacturing, Inc., Civ. App. 04-1116, 2004 U.S. App. LEXIS 18265, *13-14 (Fed. Cir. Aug. 27, 2004) (unpublished). The term “substantially” is commonly used by claim drafters to indicate approximation. See Cordis Corp. v. Medtronic AVE Inc., 339 F.3d 1352, 1360 (Fed. Cir. 2003) (“The patents do not set out any numerical standard by which to determine whether the thickness of the wall surface is ‘substantially uniform.’ The term ‘substantially,’ as used in this context, denotes approximation. Thus, the walls must be of largely or approximately uniform thickness.”); see also Deering Precision Instruments, LLC v. Vector Distribution Sys., Inc., 347 F.3d 1314, 1322 (Fed. Cir. 2003); Epcon Gas Sys., Inc. v. Bauer Compressors, Inc., 279 F.3d 1022, 1031 (Fed. Cir. 2002). We find that the term “substantially” was used in just such a manner in the claims of the patents-in-suit: “substantially uniform wall thickness” denotes a wall thickness with approximate uniformity.

It should also be noted that such words of approximation as contemplated in the foregoing clearly limits the scope of claims such as saying ‘generally parallel’ such that the adverb ‘generally’ does not broaden the meaning of parallel. Accordingly, it is well settled that such words of approximation as contemplated in the foregoing (e.g., like the phrase ‘generally parallel’) envisions some amount of deviation from perfection (e.g., not exactly parallel), and that such words of approximation as contemplated in the foregoing are descriptive terms commonly used in patent claims to avoid a strict numerical boundary to the specified parameter. To the extent that the plain language of the claims relying on such words of approximation as contemplated in the foregoing are clear and uncontradicted by anything in the written description herein or the figures thereof, it is improper to rely upon the present written description, the figures, or the prosecution history to add limitations to any of the claim of the present invention with respect to such words of approximation as contemplated in the foregoing. That is, under such circumstances, relying on the written description and prosecution history to reject the ordinary and customary meanings of the words themselves is impermissible. See, for example, Liquid Dynamics Corp. v. Vaughan Co., 355 F.3d 1361, 69 USPQ2d 1595, 1600-01 (Fed. Cir. 2004). The plain language of phrase 2 requires a “substantial helical flow.” The term “substantial” is a meaningful modifier implying “approximate,” rather than “perfect.” In Cordis Corp. v. Medtronic AVE, Inc., 339 F.3d 1352, 1361 (Fed. Cir. 2003), the district court imposed a precise numeric constraint on the term “substantially uniform thickness.” We noted that the proper interpretation of this term was “of largely or approximately uniform thickness” unless something in the prosecution history imposed the “clear and unmistakable disclaimer” needed for narrowing beyond this simple-language interpretation. Id. In Anchor Wall Systems v. Rockwood Retaining Walls, Inc., 340 F.3d 1298, 1311 (Fed. Cir. 2003)” Id. at 1311. Similarly, the plain language of Claim 1 requires neither a perfectly helical flow nor a flow that returns precisely to the center after one rotation (a limitation that arises only as a logical consequence of requiring a perfectly helical flow).

The reader should appreciate that case law generally recognizes a dual ordinary meaning of such words of approximation, as contemplated in the foregoing, as connoting a term of approximation or a term of magnitude; e.g., see Deering Precision Instruments, L.L.C. v. Vector Distrib. Sys., Inc., 347 F.3d 1314, 68 USPQ2d 1716, 1721 (Fed. Cir. 2003), cert. denied, 124 S. Ct. 1426 (2004) where the court was asked to construe the meaning of the term “substantially” in a patent claim. Also see Epcon, 279 F.3d at 1031 (“The phrase ‘substantially constant’ denotes language of approximation, while the phrase ‘substantially below’ signifies language of magnitude, i.e., not insubstantial.”). Also, see, e.g., Epcon Gas Sys., Inc. v. Bauer Compressors, Inc., 279 F.3d 1022 (Fed. Cir. 2002) (construing the terms “substantially constant” and “substantially below”); Zodiac Pool Care, Inc. v. Hoffinger Indus., Inc., 206 F.3d 1408 (Fed. Cir. 2000) (construing the term “substantially inward”); York Prods., Inc. v. Cent. Tractor Farm & Family Ctr., 99 F.3d 1568 (Fed. Cir. 1996) (construing the term “substantially the entire height thereof”); Tex. Instruments Inc. v. Cypress Semiconductor Corp., 90 F.3d 1558 (Fed. Cir. 1996) (construing the term “substantially in the common plane”). In conducting their analysis, the court instructed to begin with the ordinary meaning of the claim terms to one of ordinary skill in the art. Prima Tek, 318 F.3d at 1148. Reference to dictionaries and our cases indicates that the term “substantially” has numerous ordinary meanings. As the district court stated, “substantially” can mean “significantly” or “considerably.” The term “substantially” can also mean “largely” or “essentially.” Webster's New 20th Century Dictionary 1817 (1983).

Words of approximation, as contemplated in the foregoing, may also be used in phrases establishing approximate ranges or limits, where the end points are inclusive and approximate, not perfect; e.g., see AK Steel Corp. v. Sollac, 344 F.3d 1234, 68 USPQ2d 1280, 1285 (Fed. Cir. 2003) where it where the court said [W]e conclude that the ordinary meaning of the phrase “up to about 10%” includes the “about 10%” endpoint. As pointed out by AK Steel, when an object of the preposition “up to” is nonnumeric, the most natural meaning is to exclude the object (e.g., painting the wall up to the door). On the other hand, as pointed out by Sollac, when the object is a numerical limit, the normal meaning is to include that upper numerical limit (e.g., counting up to ten, seating capacity for up to seven passengers). Because we have here a numerical limit —“about 10%”— the ordinary meaning is that that endpoint is included.

In the present specification and claims, a goal of employment of such words of approximation, as contemplated in the foregoing, is to avoid a strict numerical boundary to the modified specified parameter, as sanctioned by Pall Corp. v. Micron Separations, Inc., 66 F.3d 1211, 1217, 36 USPQ2d 1225, 1229 (Fed. Cir. 1995) where it states “It is well established that when the term “substantially” serves reasonably to describe the subject matter so that its scope would be understood by persons in the field of the invention, and to distinguish the claimed subject matter from the prior art, it is not indefinite.” Likewise see Verve LLC v. Crane Cams Inc., 311 F.3d 1116, 65 USPQ2d 1051, 1054 (Fed. Cir. 2002). Expressions such as “substantially” are used in patent documents when warranted by the nature of the invention, in order to accommodate the minor variations that may be appropriate to secure the invention. Such usage may well satisfy the charge to “particularly point out and distinctly claim” the invention, 35 U.S.C. § 112, and indeed may be necessary in order to provide the inventor with the benefit of his invention. In Andrew Corp. v. Gabriel Elecs. Inc., 847 F.2d 819, 821-22, 6 USPQ2d 2010, 2013 (Fed. Cir. 1988) the court explained that usages such as “substantially equal” and “closely approximate” may serve to describe the invention with precision appropriate to the technology and without intruding on the prior art. The court again explained in Ecolab Inc. v. Envirochem, Inc., 264 F.3d 1358, 1367, 60 USPQ2d 1173, 1179 (Fed. Cir. 2001) that “like the term ‘about,’ the term ‘substantially’ is a descriptive term commonly used in patent claims to ‘avoid a strict numerical boundary to the specified parameter, see Ecolab Inc. v. Envirochem Inc., 264 F.3d 1358, 60 USPQ2d 1173, 1179 (Fed. Cir. 2001) where the court found that the use of the term “substantially” to modify the term “uniform” does not render this phrase so unclear such that there is no means by which to ascertain the claim scope.

Similarly, other courts have noted that like the term “about,” the term “substantially” is a descriptive term commonly used in patent claims to “avoid a strict numerical boundary to the specified parameter.”; e.g., see Pall Corp. v. Micron Seps., 66 F.3d 1211, 1217, 36 USPQ2d 1225, 1229 (Fed. Cir. 1995); see, e.g., Andrew Corp. v. Gabriel Elecs. Inc., 847 F.2d 819, 821-22, 6 USPQ2d 2010, 2013 (Fed. Cir. 1988) (noting that terms such as “approach each other,” “close to,” “substantially equal,” and “closely approximate” are ubiquitously used in patent claims and that such usages, when serving reasonably to describe the claimed subject matter to those of skill in the field of the invention, and to distinguish the claimed subject matter from the prior art, have been accepted in patent examination and upheld by the courts). In this case, “substantially” avoids the strict 100% nonuniformity boundary.

Indeed, the foregoing sanctioning of such words of approximation, as contemplated in the foregoing, has been established as early as 1939, see Ex parte Mallory, 52 USPQ 297, 297 (Pat. Off. Bd. App. 1941) where, for example, the court said, “the claims specify that the film is “substantially” eliminated and for the intended purpose, it is believed that the slight portion of the film which may remain is negligible. We are of the view, therefore, that the claims may be regarded as sufficiently accurate.” Similarly, In re Hutchison, 104 F.2d 829, 42 USPQ 90, 93 (C.C.P.A. 1939) the court said “It is realized that “substantial distance” is a relative and somewhat indefinite term, or phrase, but terms and phrases of this character are not uncommon in patents in cases where, according to the art involved, the meaning can be determined with reasonable clearness.”

Hence, for at least the forgoing reason, Applicants submit that it is improper for any examiner to hold as indefinite any claims of the present patent that employ any words of approximation.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will be described in detail below with reference to embodiments thereof as illustrated in the accompanying drawings.

References to a “device,” an “apparatus,” a “system,” etc., in the preamble of a claim should be construed broadly to mean “any structure meeting the claim terms” exempt for any specific structure(s)/type(s) that has/(have) been explicitly disavowed or excluded or admitted/implied as prior art in the present specification or incapable of enabling an object/aspect/goal of the invention. Furthermore, where the present specification discloses an object, aspect, function, goal, result, or advantage of the invention that a specific prior art structure and/or method step is similarly capable of performing yet in a very different way, the present invention disclosure is intended to and shall also implicitly include and cover additional corresponding alternative embodiments that are otherwise identical to that explicitly disclosed except that they exclude such prior art structure(s)/step(s), and shall accordingly be deemed as providing sufficient disclosure to support a corresponding negative limitation in a claim claiming such alternative embodiment(s), which exclude such very different prior art structure(s)/step(s) way(s).

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this Application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present Application or of any further Application derived therefrom.

References to “one embodiment,” “an embodiment,” “example embodiment,” “various embodiments,” “some embodiments,” “embodiments of the invention,” etc., may indicate that the embodiment(s) of the invention so described may include a particular feature, structure, or characteristic, but not every possible embodiment of the invention necessarily includes the particular feature, structure, or characteristic. Further, repeated use of the phrase “in one embodiment,” or “in an exemplary embodiment,” “an embodiment,” do not necessarily refer to the same embodiment, although they may. Moreover, any use of phrases like “embodiments” in connection with “the invention” are never meant to characterize that all embodiments of the invention must include the particular feature, structure, or characteristic, and should instead be understood to mean “at least some embodiments of the invention” includes the stated particular feature, structure, or characteristic.

References to “user”, or any similar term, as used herein, may mean a human or non-human user thereof. Moreover, “user”, or any similar term, as used herein, unless expressly stipulated otherwise, is contemplated to mean users at any stage of the usage process, to include, without limitation, direct user(s), intermediate user(s), indirect user(s), and end user(s). The meaning of “user”, or any similar term, as used herein, should not be otherwise inferred or induced by any pattern(s) of description, embodiments, examples, or referenced prior-art that may (or may not) be provided in the present patent.

References to “end user”, or any similar term, as used herein, is generally intended to mean late stage user(s) as opposed to early stage user(s). Hence, it is contemplated that there may be a multiplicity of different types of “end user” near the end stage of the usage process. Where applicable, especially with respect to distribution channels of embodiments of the invention comprising consumed retail products/services thereof (as opposed to sellers/vendors or Original Equipment Manufacturers), examples of an “end user” may include, without limitation, a “consumer”, “buyer”, “customer”, “purchaser”, “shopper”, “enjoyer”, “viewer”, or individual person or non-human thing benefiting in any way, directly or indirectly, from use of or interaction, with some aspect of the present invention.

In some situations, some embodiments of the present invention may provide beneficial usage to more than one stage or type of usage in the foregoing usage process. In such cases where multiple embodiments targeting various stages of the usage process are described, references to “end user”, or any similar term, as used therein, are generally intended to not include the user that is the furthest removed, in the foregoing usage process, from the final user therein of an embodiment of the present invention.

Where applicable, especially with respect to retail distribution channels of embodiments of the invention, intermediate user(s) may include, without limitation, any individual person or non-human thing benefiting in any way, directly or indirectly, from use of, or interaction with, some aspect of the present invention with respect to selling, vending, Original Equipment Manufacturing, marketing, merchandising, distributing, service providing, and the like thereof.

References to “person”, “individual”, “human”, “a party”, “animal”, “creature”, or any similar term, as used herein, even if the context or particular embodiment implies living user, maker, or participant, it should be understood that such characterizations are sole by way of example, and not limitation, in that it is contemplated that any such usage, making, or participation by a living entity in connection with making, using, and/or participating, in any way, with embodiments of the present invention may be substituted by such similar performed by a suitably configured non-living entity, to include, without limitation, automated machines, robots, humanoids, computational systems, information processing systems, artificially intelligent systems, and the like. It is further contemplated that those skilled in the art will readily recognize the practical situations where such living makers, users, and/or participants with embodiments of the present invention may be in whole, or in part, replaced with such non-living makers, users, and/or participants with embodiments of the present invention. Likewise, when those skilled in the art identify such practical situations where such living makers, users, and/or participants with embodiments of the present invention may be in whole, or in part, replaced with such non-living makers, it will be readily apparent in light of the teachings of the present invention how to adapt the described embodiments to be suitable for such non-living makers, users, and/or participants with embodiments of the present invention. Thus, the invention is thus to also cover all such modifications, equivalents, and alternatives falling within the spirit and scope of such adaptations and modifications, at least in part, for such non-living entities.

Headings provided herein are for convenience and are not to be taken as limiting the disclosure in any way.

The enumerated listing of items does not imply that any or all of the items are mutually exclusive, unless expressly specified otherwise.

It is understood that the use of specific component, device and/or parameter names are for example only and not meant to imply any limitations on the invention. The invention may thus be implemented with different nomenclature/terminology utilized to describe the mechanisms/units/structures/components/devices/parameters herein, without limitation. Each term utilized herein is to be given its broadest interpretation given the context in which that term is utilized.

Terminology. The following paragraphs provide definitions and/or context for terms found in this disclosure (including the appended claims):

“Comprising.” This term is open-ended. As used in the appended claims, this term does not foreclose additional structure or steps. Consider a claim that recites: “A memory controller comprising a system cache ” Such a claim does not foreclose the memory controller from including additional components (e.g., a memory channel unit, a switch).

“Configured To.” Various units, circuits, or other components may be described or claimed as “configured to” perform a task or tasks. In such contexts, “configured to” or “operable for” is used to connote structure by indicating that the mechanisms/units/circuits/components include structure (e.g., circuitry and/or mechanisms) that performs the task or tasks during operation. As such, the mechanisms/unit/circuit/component can be said to be configured to (or be operable) for perform(ing) the task even when the specified mechanisms/unit/circuit/component is not currently operational (e.g., is not on). The mechanisms/units/circuits/components used with the “configured to” or “operable for” language include hardware—for example, mechanisms, structures, electronics, circuits, memory storing program instructions executable to implement the operation, etc. Reciting that a mechanism/unit/circuit/component is “configured to” or “operable for” perform(ing) one or more tasks is expressly intended not to invoke 35 U.S.C. sctn. 112, sixth paragraph, for that mechanism/unit/circuit/component. “Configured to” may also include adapting a manufacturing process to fabricate devices or components that are adapted to implement or perform one or more tasks.

“Based On.” As used herein, this term is used to describe one or more factors that affect a determination. This term does not foreclose additional factors that may affect a determination. That is, a determination may be solely based on those factors or based, at least in part, on those factors. Consider the phrase “determine A based on B.” While B may be a factor that affects the determination of A, such a phrase does not foreclose the determination of A from also being based on C. In other instances, A may be determined based solely on B.

The terms “a”, “an” and “the” mean “one or more”, unless expressly specified otherwise.

Unless otherwise indicated, all numbers expressing conditions, concentrations, dimensions, and so forth used in the specification and claims are to be understood as being modified in all instances by the term “about.” Accordingly, unless indicated to the contrary, the numerical parameters set forth in the following specification and attached claims are approximations that may vary depending at least upon a specific analytical technique.

The term “comprising,” which is synonymous with “including,” “containing,” or “characterized by” is inclusive or open-ended and does not exclude additional, unrecited elements or method steps. “Comprising” is a term of art used in claim language which means that the named claim elements are essential, but other claim elements may be added and still form a construct within the scope of the claim.

As used herein, the phase “consisting of” excludes any element, step, or ingredient not specified in the claim. When the phrase “consists of” (or variations thereof) appears in a clause of the body of a claim, rather than immediately following the preamble, it limits only the element set forth in that clause; other elements are not excluded from the claim as a whole. As used herein, the phase “consisting essentially of” and “consisting of” limits the scope of a claim to the specified elements or method steps, plus those that do not materially affect the basis and novel characteristic(s) of the claimed subject matter (see Norian Corp. v Stryker Corp., 363 F.3d 1321, 1331-32, 70 USPQ2d 1508, Fed. Cir. 2004). Moreover, for any claim of the present invention which claims an embodiment “consisting essentially of” or “consisting of” a certain set of elements of any herein described embodiment it shall be understood as obvious by those skilled in the art that the present invention also covers all possible varying scope variants of any described embodiment(s) that are each exclusively (i.e., “consisting essentially of”) functional subsets or functional combination thereof such that each of these plurality of exclusive varying scope variants each consists essentially of any functional subset(s) and/or functional combination(s) of any set of elements of any described embodiment(s) to the exclusion of any others not set forth therein. That is, it is contemplated that it will be obvious to those skilled how to create a multiplicity of alternate embodiments of the present invention that simply consisting essentially of a certain functional combination of elements of any described embodiment(s) to the exclusion of any others not set forth therein, and the invention thus covers all such exclusive embodiments as if they were each described herein.

With respect to the terms “comprising,” “consisting of,” and “consisting essentially of ” where one of these three terms is used herein, the presently disclosed and claimed subject matter may include the use of either of the other two terms. Thus in some embodiments not otherwise explicitly recited, any instance of “comprising” may be replaced by “consisting of” or, alternatively, by “consisting essentially of”, and thus, for the purposes of claim support and construction for “consisting of” format claims, such replacements operate to create yet other alternative embodiments “consisting essentially of” only the elements recited in the original “comprising” embodiment to the exclusion of all other elements.

Devices or system modules that are in at least general communication with each other need not be in continuous communication with each other, unless expressly specified otherwise. In addition, devices or system modules that are in at least general communication with each other may communicate directly or indirectly through one or more intermediaries.

A description of an embodiment with several components in communication with each other does not imply that all such components are required. On the contrary a variety of optional components are described to illustrate the wide variety of possible embodiments of the present invention.

As is well known to those skilled in the art many careful considerations and compromises typically must be made when designing for the optimal manufacture of a commercial implementation any system, and in particular the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

In one embodiment, a method of using a porous, biocompatible, biodegradable, resorbable, non-toxic, and polymerizable adhesive composition is provided. In one embodiment, the adhesive composition may be capable of adhering to bone structure, implants, devices, teeth, bone graft materials, and membranes. In one embodiment, the device may include, but not be limited to metal screws, pins, rods, or plates, and the like devices that may be used in bone structure repair.

In one embodiment, the method includes a step of applying a porous, biocompatible, biodegradable, resorbable, non-toxic, and polymerizable adhesive composition to substrate bone repair regions including but not limited to bone structure, implant or device surface if used, tooth surface if present, bone graft materials if utilized, and to the barrier membrane that physically separates the osseous tissue from the soft tissue.

In one embodiment, the use of an adhesive in a chemical composition used as a bone graft material or and as bone substitutes or and as a barrier membrane, may assist in bonding the surrounding bone, implant, device, tooth, together in the desired form preserving the space volume required for bone regeneration and preventing soft tissue invasion of the treated site during the healing period. In one embodiment, the use of an adhesive in a chemical composition used with bone graft material, may assist in bonding the bone graft materials together and to the surrounding bone, implant, device, tooth, or barrier membrane. In one embodiment, the use of the adhesive in a chemical composition used with bone graft material, may render the bone graft material more flexible, and it may be easier to shape the bone graft materials to a desired form and retain the bone graft material in that form during the healing period. In one embodiment, the use of adhesive in a chemical composition over a barrier membrane may keep the membrane in place, and may prevent soft tissues from growing into the bone graft material and the osseous tissues.

In one embodiment, the pores allow for free transport of fluids, i.e., gases and liquids through the pores in the adhesive. The pores may also assist in neovascularization and bone formation in the substrate bone repair region. In one embodiment, the pores may have an average diameter in a range of from about 100 microns to about 1000 microns. In another embodiment, the pores may have an average diameter in a range of from about 150 microns to about 950 microns. In yet another embodiment, the pores may have an average diameter in a range of from about 200 microns to about 900 microns. In one embodiment, the pores may be multidimensional, i.e., may include pores with varying mean sizes.

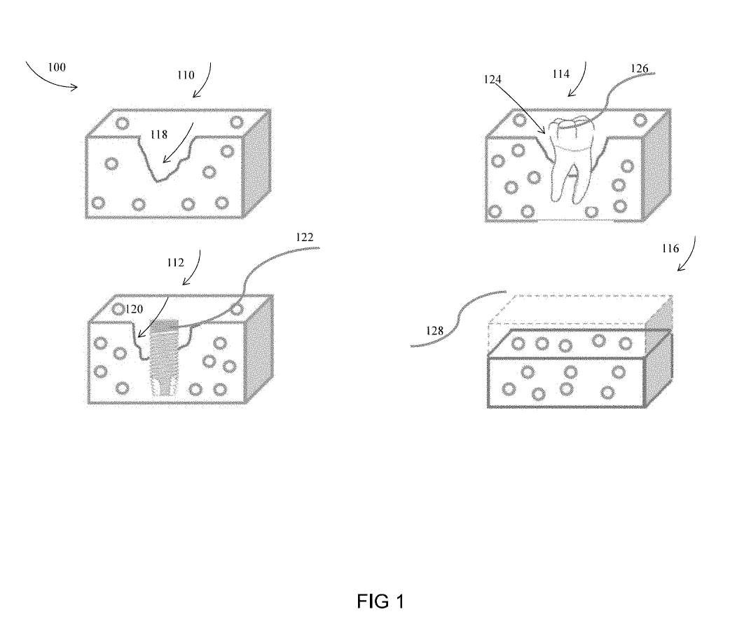

Referring to FIG. 1 is illustrated perspective views 100 of substrate bone repair regions, in accordance with an embodiment of the present invention. As shown in FIG. 1, view 110 shows a bone defect, bone hole, or bone fracture 118, view 112 shows a bone defect 120 around an implant or a device fitted in the bone 122, view 114 shows a bone defect 124 around a tooth 126, and view 116 shows a bone augmentation 128. It may be appreciated by a person with ordinary skill in the art, in light of and in accordance with the teachings of the present invention that the procedures described herein in view 110, 112, 114, and 116 may be performed by a trained medical practitioner. As used herein, bone augmentation is a term that describes a variety of procedures used to “build” bone. These procedures may typically involve grafting (adding) bone or bone like materials to a substrate bone repair region in any part of the body.

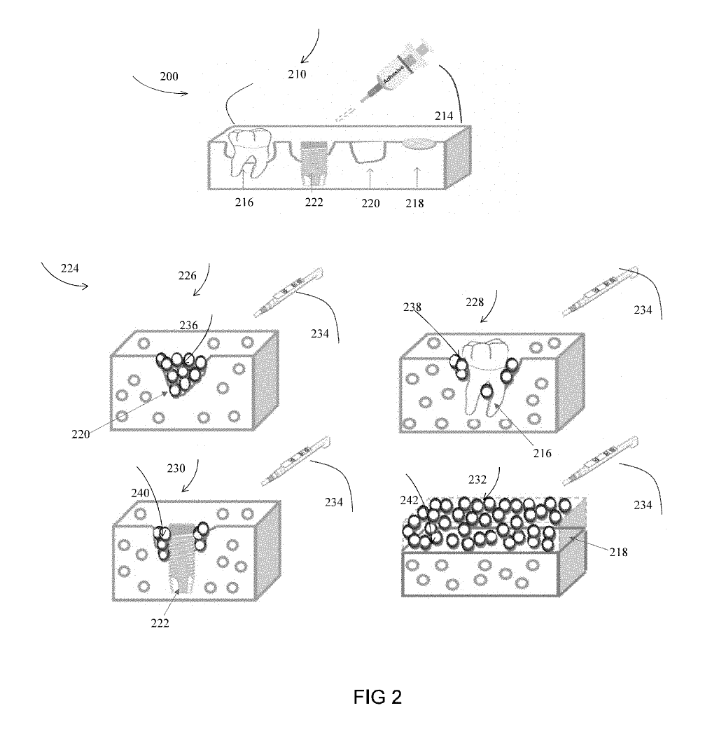

Referring to FIG. 2 is illustrated a method 200 of applying an adhesive composition on a substrate bone repair region in accordance with an embodiment of the present invention. In one embodiment, the method 200 may comprise a first step 210 wherein the adhesive composition 214 may be applied directly to the substrate bone repair region. In various embodiments, the substrate bone repair region may include, but not be limited to, a bone defect region 220, an implant or device 222 in a substrate bone repair region, a bone augmentation 218 in a substrate bone repair region, a tooth 216, and the like. In one embodiment, the polymerization of the adhesive composition on the surface of substrate bone repair region may be achieved either chemically or by curing (using heat or light) as shown in the substrate bone repair regions. Accordingly, in a second step 224, the adhesive composition 214 in the substrate bone repair regions may be cured 234 as shown in view 226 in the bone defect area 220 by 236, in view 230 surrounding the implant or a device 222 indicated by 240, in view 228 surrounding the tooth 216 by 238, and in view 232 over the bone augmentation 218 by 242. In certain embodiments, the adhesive composition may be mixed with a bone graft material. In certain embodiments, the adhesive composition may be applied to a barrier membrane and this may be applied on the substrate bone repair region where the adhesive composition has been previously applied.

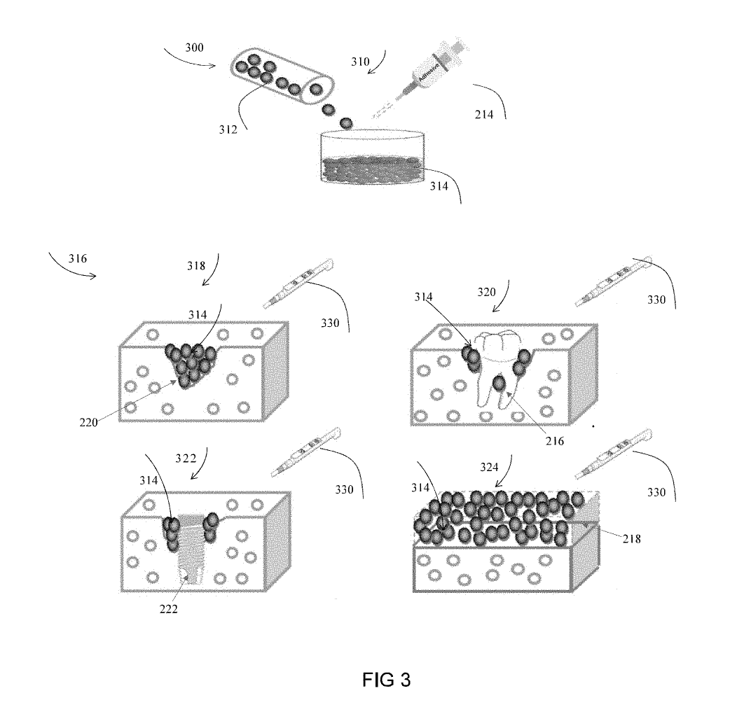

Referring to FIG. 3, is illustrated a method 300 of using an adhesive composition in combination with a bone graft material, in accordance with an embodiment of the present invention. The method 300 may comprise a first step 310 wherein an adhesive composition 214 may be mixed with a bone graft material 312 to provide an adhesive chemical composition 314. In a second step 316 the chemical composition 314 formed by mixing the bone graft material 312 with the adhesive composition 214 as shown in view 316 on the substrate bone repair regions, i.e., in view 318 in the bone defect area 220, in view 322 surrounding the implant or a device 222, in view 320 surrounding the tooth 216, and in view 324 over the bone augmentation 218. As shown in FIG. 3 the chemical composition 314 occupies the gaps or blank spaces in the substrate bone repair regions as show in view 318, 320, 322, and 324. In one embodiment, the polymerization of the adhesive in the bone graft material and the adhesive on the substrate bone repair region may be achieved either chemically or by curing (using heat or light) from curing device 330.

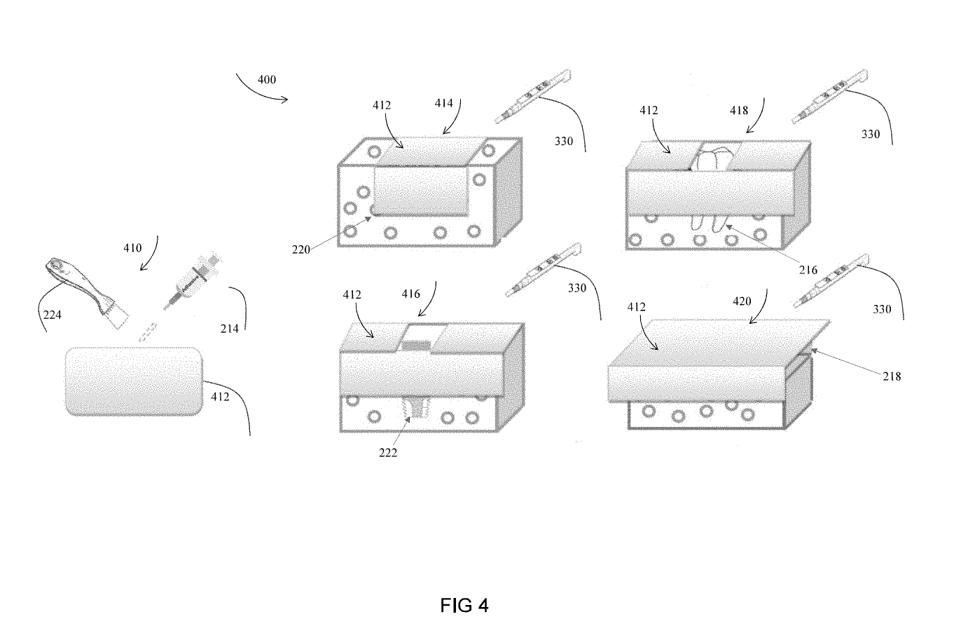

In one embodiment, a barrier membrane may be used to prevent the soft tissues invasion into the region where either the adhesive material 214 or the chemical composition 314 comprising the bone graft material 312 and the adhesive composition 214 has been applied. As shown in FIG. 4, if a membrane 412 is being used, then in a step 410, the adhesive composition 214 may be applied 224 to the barrier membrane 412 before the barrier membrane 412 may be placed on the substrate bone repair region where either the adhesive material 214 or the chemical composition 314 comprising the bone graft material 312 and the adhesive composition 214 has been applied. As shown in FIG. 4 the membrane 412 is placed over the substrate bone repair regions, i.e., in view 414 over the bone defect area 220, in view 416 over the implant or a device 222, in view 418 over the tooth 216, and in view 420 over the bone augmentation 218. In one embodiment, the polymerization of the adhesive in the bone graft material and the adhesive on the surface of the membrane may be achieved either chemically or by curing (using heat or light) from curing device 330. It may be appreciated by a person with ordinary skill in the art, in light of and in accordance with the teachings of the present invention that the use of a membrane may be optional in certain embodiments, when a layer of the adhesive may be used on top of the bone graft. In certain embodiments, it may be cost effective to use the membrane, particularly when relatively larger surface areas need to be covered.

In one embodiment, the adhesive may include but is not limited to collagen based or protein based adhesives, for example, Bio Glue™. In one embodiment, the adhesive may be resorbable, i.e., biodegradable. The adhesive may be broken down by the body with time. In one embodiment, the body may take a time in a range of from about three months to about six months to degrade the adhesive material.

In one embodiment, suitable bone graft material may include, but not be limited to, bone, a synthetic material, a growth factor, a cell, or a combination thereof. Suitable bones may include, but are not limited to autograft materials, i.e., bone collected from the individual receiving the graft; autologous bone, autologous bone particulate, allogenic bones i.e., allograft materials, i.e., bone collected from human donors or human cadavers; xenograft materials, i.e., bone collected from a species other than humans, and bone chips. Suitable synthetic materials may include but are not limited to, ceramics, hydroxyapatite, tricalcium phosphate, calcium phosphate, calcium sulfate, polymer based bone-graft substitutes, composite materials, and the like. Suitable examples of allograft materials may include, but not be limited to, Puros®, DBX®, EnCore®, Raptos®, PentOS®, Maxxeus®, etc. Suitable examples of xenograft materials may include, but not be limited to, Endobone®, BioOss®, Instradent®, Symbios OsteoGraf N®, etc. Suitable examples of synthetic material may include, but not be limited to, IngeniOs®, Bioplant®, NovaBone®, Perioglas®, DentoGen®, etc. Suitable growth factors include, but are not limited to, is bone morphogenic protein (BMP), a BMP mimetic, recombinant human bone morphogenic protein 2 (rhBMP-2), platelet-rich plasma (PRP), transforming growth factor-beta (TGF-beta), platelet-derived growth factor (PDGF), recombinant human platelet derived growth factor (rhPDGF), insulin-like growth factor, fibroblast growth factor, a xenographic bone protein, a growth differentiation factor, calcitonin, and a calcitonin mimetic.

Suitable living cells include, but are not limited to, stem cell, a bone marrow cell, a mesenchymal stem cell, and a combination thereof.

As used herein the term “resorbable” may be referred to an adhesive composition that can be broken down and assimilated back in the body.

As used herein the term “biodegradable” may be referred to an adhesive composition that is susceptible of beak-down into simpler components by biological processes in the body.

As used herein the term “biocompatible” may be referred to an adhesive composition that is not harmful to living tissue.

As used herein the term “non-toxic” may be referred to an adhesive composition that not-poisonous or toxic.

As used herein the term “augmentation” may be referred to as building a bone surface on a substrate bone repair region or increasing the volume of the substrate bone repair region.

In one embodiment, suitable adhesives may include, but not be limited to, Bio Glue®, which includes purified bovine serum albumin (BSA) and glutaraldehyde.

In one embodiment, adhesive may be used with bone graft material. In one embodiment, the adhesive and bone graft material may be used in a ratio in a range of from about adhesive:bone graft material: 50:50. In another embodiment, the adhesive and bone graft material may be used in a ratio in a range of from about adhesive:bone graft material: 30:70.In yet another embodiment, the adhesive and bone graft material may be used in a ratio in a range of from about adhesive:bone graft material: 20:80. In certain embodiments, adhesive may be used independent of the bone graft material.

In one embodiment, the polymerizable composition may require a curing time in a range of about 20 seconds to about 120 seconds based on the method of curing employed, i.e., curing in the presence of light or chemical curing. It may be appreciated by a person with ordinary skill in the art, in light of and in accordance with the teachings of the present invention that the light used for curing or the chemical used for curing may be dependent on the type of adhesive chosen in the polymerizable composition.

Those skilled in the art will readily recognize, in light of and in accordance with the teachings of the present invention, that any of the foregoing steps may be suitably replaced, reordered, removed and additional steps may be inserted depending upon the needs of the particular application. Moreover, the prescribed method steps of the foregoing embodiments may be implemented using any physical and/or hardware system that those skilled in the art will readily know is suitable in light of the foregoing teachings. For any method steps described in the present application that can be carried out on a computing machine, a typical computer system can, when appropriately configured or designed, serve as a computer system in which those aspects of the invention may be embodied. Thus, the present invention is not limited to any particular tangible means of implementation.

Accordingly, the invention described herein includes a method for bone preservation, bone regeneration to repair defects, and bone augmentation. The method consists of applying a porous, biocompatible, biodegradable, resorbable, non-toxic, polymerizable adhesive composition to a substrate bone structure, implant device surface if used, tooth if present, bone graft materials if utilized, and to the barrier membrane that separates the osseous tissue from the soft tissue. The adhesive may bond to the substrate bone structure, implant or device surface if used, tooth surface if present, bone graft materials if utilized and barrier membrane. In one embodiment, the adhesive may hold the bone graft materials together and may help shape the bone graft materials to the desired form, adhere the bone graft materials to the substrate bone structure, implant or device surface, or tooth on one side and to the barrier membrane that cover the bone graft materials on the other side and keep them all in place and in the desired form preserving the space required for bone generation. In one embodiment, the adhesive may work as a bone graft material preserving the space for bone regeneration and may act as membrane preventing the soft tissues invasion into the defect and forms a chamber to guide the bone regeneration process.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

It is noted that according to USA law 35 USC § 112 (1), all claims must be supported by sufficient disclosure in the present patent specification, and any material known to those skilled in the art need not be explicitly disclosed. However, 35 USC § 112 (6) requires that structures corresponding to functional limitations interpreted under 35 USC § 112 (6) must be explicitly disclosed in the patent specification. Moreover, the USPTO's Examination policy of initially treating and searching prior art under the broadest interpretation of a “mean for” claim limitation implies that the broadest initial search on 112(6) functional limitation would have to be conducted to support a legally valid Examination on that USPTO policy for broadest interpretation of “mean for” claims. Accordingly, the USPTO will have discovered a multiplicity of prior art documents including disclosure of specific structures and elements which are suitable to act as corresponding structures to satisfy all functional limitations in the below claims that are interpreted under 35 USC § 112 (6) when such corresponding structures are not explicitly disclosed in the foregoing patent specification. Therefore, for any invention element(s)/structure(s) corresponding to functional claim limitation(s), in the below claims interpreted under 35 USC § 112 (6), which is/are not explicitly disclosed in the foregoing patent specification, yet do exist in the patent and/or non-patent documents found during the course of USPTO searching, Applicant(s) incorporate all such functionally corresponding structures and related enabling material herein by reference for the purpose of providing explicit structures that implement the functional means claimed. Applicant(s) request(s) that fact finders during any claims construction proceedings and/or examination of patent allowability properly identify and incorporate only the portions of each of these documents discovered during the broadest interpretation search of 35 USC § 112 (6) limitation, which exist in at least one of the patent and/or non-patent documents found during the course of normal USPTO searching and or supplied to the USPTO during prosecution. Applicant(s) also incorporate by reference the bibliographic citation information to identify all such documents comprising functionally corresponding structures and related enabling material as listed in any PTO Form-892 or likewise any information disclosure statements (IDS) entered into the present patent application by the USPTO or Applicant(s) or any 3^{rd} parties. Applicant(s) also reserve its right to later amend the present application to explicitly include citations to such documents and/or explicitly include the functionally corresponding structures which were incorporate by reference above.

Thus, for any invention element(s)/structure(s) corresponding to functional claim limitation(s), in the below claims, that are interpreted under 35 USC § 112 (6), which is/are not explicitly disclosed in the foregoing patent specification, Applicant(s) have explicitly prescribed which documents and material to include the otherwise missing disclosure, and have prescribed exactly which portions of such patent and/or non-patent documents should be incorporated by such reference for the purpose of satisfying the disclosure requirements of 35 USC § 112 (6). Applicant(s) note that all the identified documents above which are incorporated by reference to satisfy 35 USC § 112 (6) necessarily have a filing and/or publication date prior to that of the instant application, and thus are valid prior documents to incorporated by reference in the instant application.

Having fully described at least one embodiment of the present invention, other equivalent or alternative methods of implementing a method for using an adhesive composition in bone preservation according to the present invention will be apparent to those skilled in the art. Various aspects of the invention have been described above by way of illustration, and the specific embodiments disclosed are not intended to limit the invention to the particular forms disclosed. The particular implementation of the method for bone preservation may vary depending upon the particular context or application. By way of example, and not limitation, the method for bone preservation described in the foregoing were principally directed to a method for bone preservation implementations; however, similar techniques may instead be applied to a method for using an adhesive composition in bone preservation, which implementations of the present invention are contemplated as within the scope of the present invention. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

Claim elements and steps herein may have been numbered and/or lettered solely as an aid in readability and understanding. Any such numbering and lettering in itself is not intended to and should not be taken to indicate the ordering of elements and/or steps in the claims.

The corresponding structures, materials, acts, and equivalents of all means or step plus function elements in the claims below are intended to include any structure, material, or act for performing the function in combination with other claimed elements as specifically claimed.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

The Abstract is provided to comply with 37 C.F.R. Section 1.72(b) requiring an abstract that will allow the reader to ascertain the nature and gist of the technical disclosure. That is, the Abstract is provided merely to introduce certain concepts and not to identify any key or essential features of the claimed subject matter. It is submitted with the understanding that it will not be used to limit or interpret the scope or meaning of the claims.

The following claims are hereby incorporated into the detailed description, with each claim standing on its own as a separate embodiment.

1. A method comprising the steps of:
applying an adhesive composition to a substrate bone repair region, said adhesive composition comprises at least a bovine serum albumin or collagen based adhesive;
wherein said bovine serum albumin or collagen based adhesive composition comprises at least a porous, biocompatible, biodegradable, resorbable, or polymerizable adhesive composition;
wherein said substrate bone repair region comprises at least one of a bone structure, an implant device surface, and a tooth surface;
curing said bovine serum albumin or collagen based adhesive composition applied to said substrate bone repair region with a curing device employing at least light curing or chemical curing;
wherein said curing step is configured to generate a polymerized bovine serum albumin or collagen based adhesive composition on said substrate bone repair region; and
wherein said curing step is further configured to be operable for at least one of repairing said bone structure, filling gaps between said bone structure or implant device surface, and filling gaps between said bone structure or tooth surface.

2. The method of claim 1, wherein said bovine serum albumin or collagen based adhesive composition comprises pores, in which said pores are configured to be operable for allowing free transport of fluids through the adhesive.

3. The method of claim 2, wherein said pores have an average diameter in a range of from about 100 microns to about 1000 microns.

4. The method claim 2, wherein said pores are further configured to be operable for assisting in neovascularization and bone formation in said substrate bone repair region.

5. The method of claim 1, wherein said resorbable bovine serum albumin or collagen based adhesive is broken down and assimilated back into the body by biological processes.

6. The method of claim 1, wherein said curing step is carried out using light.

7. The method of claim 6, wherein said curing step is carried out in a period ranging from about 10 seconds to about 60 seconds.

8. The method of claim 1, wherein said curing step is accomplished chemically.

9. The method of claim 8, where said curing step is carried out in a period ranging from about 20 seconds to about 120 seconds.

10. The method of claim 1, wherein said bovine serum albumin or collagen based adhesive composition is configured to a predetermined form and cured to preserve a space volume required for bone regeneration.

11. The method of claim 1, further comprising the steps of:
combining said bovine serum albumin or collagen based adhesive composition with a bone graft material to form an adhesive chemical composition, in which said bone graft material comprises at least one of, a synthetic material, a growth factor material, a cell material, and a combination of the cited materials;
in which said synthetic material comprises at least one of ceramics, hydroxyapatite, tricalcium phosphate, calcium phosphate, calcium sulfate, polymer based bone-graft substitutes, and composite materials;
in which said growth factors comprises at least one of a bone morphogenic protein (BMP), a BMP mimetic, a recombinant human bone morphogenic protein 2 (rhBMP-2), a platelet-rich plasma (PRP), a transforming growth factor-beta (TGF-beta), a platelet-derived growth factor (PDGF), a recombinant human platelet derived growth factor (rhPDGF), a fibroblast growth factor, a xenographic bone protein, a growth differentiation factor, a calcitonin, and a calcitonin mimetic;
in which said living cell material comprises at least one of a stem cell, a bone marrow cell, a mesenchymal stem cell, and a combination of the cited cells;
wherein said bovine serum albumin or collagen based adhesive composition in said chemical adhesive composition that is integrated with said bone graft material are configured to assist in bonding said bone graft material to said at least one of, bone structure, implant device surface, and tooth surface;
applying said combined bovine serum albumin or collagen based adhesive composition and bone graft material to said substrate bone repair region;
forming said combined bovine serum albumin or collagen based adhesive composition and bone graft material to a desired form; and
curing said adhesive chemical composition to preserve a space volume required for bone regeneration.

12. The method of claim 11, wherein said bone graft material is at least one of a bone, a synthetic material, a growth factor, and a living cell.

13. The method of claim 12, wherein said bone comprises at least one of, an autologous bone, an autologous bone particulate, an allogenic bone, a human cadaver bone, a xenograft bone, an animal bone, and bone chips.

14. The method of claim 12, wherein said synthetic material comprises at least one of, a hydroxyapatite, a calcium phosphate, a ceramics, a bioactive glass, a calcium sulfate, a polymer-based bone graft substitute, and a composite material.

15. The method of claim 12, wherein said growth factor is at least one of a bone morphogenic protein (BMP), a BMP mimetic, a recombinant human bone morphogenic protein 2 (rhBMP-2), a platelet-rich plasma (PRP), a transforming growth factor-beta (TGF-beta), a platelet-derived growth factor (PDGF), a recombinant human platelet derived growth factor (rhPDGF), a fibroblast growth factor, a xenographic bone protein, a calcitonin, and a calcitonin mimetic.

16. The method of claim 12, wherein said living cell is at least one of, a stem cell, a bone marrow cell, and a mesenchymal stem cell.

17. The method of claim 1, further comprising the steps of:
applying a barrier membrane over said substrate bone repair region, wherein said barrier membrane is configured to be operable for preventing soft tissues from growing into said substrate bone repair region;
applying said bovine serum albumin or collagen based adhesive composition over said barrier membrane to keep said barrier membrane in place, in said bone structure, implant device surface, or tooth surface.

18. The method of claim 11, further comprising the step of:
applying a barrier membrane over said combined adhesive composition and bone graft material, wherein said barrier membrane is configured to prevent soft tissues from growing into said bone graft material and adhesive composition.

19. A method comprising steps of:
combining an adhesive composition to a bone graft material, in which said adhesive composition includes at least one of, a collagen based adhesive and bovine serum albumin;
mixing said at least one of, a collagen based adhesive and bovine serum albumin adhesive composition and said bone graft material to form a chemical adhesive composition;
applying said combined at least one of, a collagen based adhesive and bovine serum albumin adhesive composition and bone graft material to a substrate bone repair region;
wherein said at least one of, a collagen based adhesive and bovine serum albumin adhesive composition comprises a porous, biocompatible, biodegradable, resorbable, or polymerizable adhesive composition; and
wherein said substrate bone repair region comprises at least one of a bone structure, an implant surface, a device surface, and a tooth surface;
curing said combined at least one of, a collagen based adhesive and bovine serum albumin adhesive composition and bone graft material;
wherein said curing step is configured to generate a polymerized at least one of, a collagen based adhesive and bovine serum albumin adhesive composition and bone graft material on the substrate bone repair region; and
wherein said ming step is configured to be operable for at least one of repairing the bone structure, filling gaps between the bone structure or implant device surface, and filling gaps in the tooth surface.

20. A method consisting of:
applying a barrier membrane to a substrate bone repair region, said substrate bone repair region comprises at least one of, a bone structure, an implant device surface, and a tooth surface;
applying an adhesive composition to said barrier membrane, said adhesive composition includes at least a bovine serum albumin and glutaraldehyde, wherein the barrier membrane is configured to separate osseous tissue from soft tissue in said substrate bone repair region, and wherein placing the barrier membrane with the adhesive composition on the substrate bone repair region is further configured to affect a repair;
wherein the bovine serum albumin and glutaraldehyde adhesive composition comprises a porous, biocompatible, biodegradable, resorbable, or polymerizable adhesive composition;
curing the mixture to produce a polymerized adhesive composition;
wherein said curing step is configured to be operable for preventing any damage to the repair by containing any movement in the substrate bone repair region using the barrier membrane with the bovine serum albumin and glutaraldehyde adhesive composition; and
wherein said curing step is further configured to be operable for repairing the bone structure, filling gaps between the bone structure or the implant device surface, or filling gaps between the bone structure or the tooth surface.

21. A method comprising steps of:
adding an adhesive composition to a bone graft material, in which said adhesive composition comprises at least a collagen based adhesive, and in which said bone graft material comprises at least one of, a bone material, a synthetic material, a growth factor, and a living cell;
mixing the collagen based adhesive composition and the bone graft material to form a mixture;
applying the collagen based adhesive composition and bone graft material mixture to a substrate bone repair region, in which said substrate bone repair region comprises at least one of, a bone structure, an implant device surface, and a tooth surface;
applying the collagen based adhesive composition to a barrier membrane, wherein the barrier membrane is configured to separate osseous tissue from soft tissue in said substrate bone repair region;
placing the barrier membrane with the collagen based adhesive composition over the collagen based adhesive composition and bone graft material mixture applied on the substrate bone repair region to affect a repair;
wherein the adhesive composition comprises a porous, biocompatible, biodegradable, resorbable, and polymerizable collagen based adhesive composition;
curing the collagen based adhesive composition present in the mixture and applied on the barrier membrane;
wherein said curing step is configured to produce a polymerized collagen based adhesive composition on the substrate bone repair region; and
wherein said curing step is further configured to be operable for at least one of repairing the bone structure, filling gaps between the bone structure or implant device surface, filling gaps between the bone structure or the tooth surface, and bone augmentation.
